(19) United States
(12) Patent Application Publication
(10) Pub. No.: US 2013/0096375 A1
(43) Pub. Date: Apr. 18, 2013

(54) HUMIDITY DETECTING METHOD AND DEVICE FOR ENDOSCOPE, AND ENDOSCOPE APPARATUS

(71) Applicant: FUJIFILM CORPORATION, Tokyo (JP)

(72) Inventors: Shozo IYAMA, Ashigarakami-gun (JP); Kenji YAMANE, Ashigarakami-gun (JP); Yasunori OHTA, Ashigarakami-gun (JP); Yasuyuki HOSONO, Ashigarakami-gun (JP); Takao OZAKI, Ashigarakami-gun (JP)

(73) Assignee: FUJIFILM CORPORATION, Tokyo (JP)

(21) Appl. No.: 13/653,980

(22) Filed: Oct. 17, 2012

(30) Foreign Application Priority Data
Oct. 18, 2011 (JP) 2011-229165

(51) Int. Cl. A61B 1/04

(52) U.S. Cl. 600/103

(57) ABSTRACT

A humidity state at a distal end portion of an endoscope internal space can be reliably estimated without enlarging the diameter of an endoscope insertion part.

In a humidity detecting device detecting humidity of an internal space of an endoscope, leakage currents of an imaging device and a peripheral circuit of an electric board disposed at a distal end portion of the internal space of the endoscope are detected and a humidity state of the internal space of the endoscope is determined based upon the detected leakage currents.

### BACKGROUND OF THE INVENTION

1. Field of the Invention

The present invention relates to humidity detecting method and device for an endoscope, and an endoscope apparatus, and in particular, to humidity detecting method and device for an endoscope, which can detect humidity in an internal space of an endoscope without enlarging a diameter of the endoscope, and an endoscope apparatus.

2. Description of the Related Art

An endoscope which has been used for a medical diagnosis must be subjected to cleaning, disinfecting, and sterilizing treatments. The disinfection is performed by dipping the endoscope in disinfectant solution in a cleaning and disinfecting apparatus or the like. In recent years, a treatment in a sterility assurance level is desired, where after an outer surface and an interior of a pipe conduit of the endoscope are cleaned by cleaning solution in a cleaning apparatus, the endoscope is put in a sterilizing package and the sterilizing treatment is performed by an autoclave sterilizing (high-temperature and high-pressure steam sterilizing) apparatus.

The endoscope has an air-tight structure, but when the endoscope is dipped in cleaning solution in a cleaning apparatus in such a state that a small hole such as a pinhole has occurred in an outer surface of the endoscope, cleaning solution may enter the internal space of the endoscope.

Further, in high-pressure vapor sterilization performed in the autoclave sterilizing apparatus, before water vapor is supplied in a chamber, air in the chamber is removed so that the chamber is pressure-reduced. Thereby, since a pressure difference between the internal space in the endoscope and the chamber occurs, which may result in damage of the endoscope, it is necessary to cause the internal space in the endoscope and the outside of the endoscope to communicate with each other, but water vapor easily enters the internal space in the endoscope. As a result, there is such a possibility that malfunction occurs in an imaging device such as CCD or an electric part such as a peripheral circuit of the imaging device provided in the internal space of the endoscope due to dampness and the imaging device does not function correctly.

In order to solve the problem, after the endoscope is cleaned and sterilized in a cleaning apparatus and in an autoclave sterilizing apparatus, the internal space in the endoscope is dehumidified and dried (for example, Japanese Patent Application Laid-Open No. 2006-136732).

Therefore, it is necessary to estimate a humidity state in the internal space of the endoscope in order to confirm the humidity state in the internal space of the endoscope which has been cleaned and sterilized in the cleaning apparatus and the autoclave sterilizing apparatus or whether or not the internal space of the endoscope has been dried sufficiently by dehumidification. Especially, it becomes important to estimate a humidity state at a distal end portion of the internal space of the endoscope where an imaging device and another electric substrate which hate dampness or moisture are disposed.

For example, in Japanese Patent Application Laid-Open No. 2005-230258, such an approach is disclosed that an absolute humidity sensor is provided at a distal end portion of an internal space of an endoscope and a measured value storing device for storing measured values measured by the absolute humidity sensor is provided therein, and a humidity state in the internal space of the endoscope is determined by capturing change between the latest measured humidity fluctuation and the measured values stored.

### SUMMARY OF THE INVENTION

However, since the imaging device such as CCD, the electric substrate, and an observation optical system are densely arranged at the distal end portion of the internal space of the endoscope, there is not a space for arranging the humidity sensor at the distal end portion. Therefore, a hard part of the distal end portion of the endoscope must be made larger in diameter or made longer in order to handle this problem, which makes an insertion portion of the endoscope larger in size and places a burden on a patient.

Therefore, when it is desired to avoid enlargement of the insertion portion of the endoscope, the humidity sensor is arranged at a portion other than the distal end portion of the endoscope, for example, a hand operation part or a connector. However, a humidity state of the distal end portion of the endoscope which should be detected originally cannot be estimated reliably by such an arrangement.

From such a background, humidity detecting method and device which can estimate a humidity state of the distal end portion of the endoscope reliably without enlarging the hard part of the distal end portion of the endoscope is demanded.

In view of these circumstances, the present invention has been made, and aims to provide humidity detecting method and device of an endoscope, and an endoscope apparatus which enable to reliably estimate a humidity state of a distal end portion of an internal space of an endoscope without size-enlarging a hard part of the distal end portion of the endoscope, an thus an imaging device such as CCD and another electric part can be prevented from malfunctioning or being damaged.

In order to achieve the above object, a humidity detecting method for detecting humidity of an internal space of an endoscope according to the present invention, comprising: a leakage current detecting step of detecting leakage current of an imaging device and/or a peripheral circuit of the imaging device arranged in the internal space of the endoscope; and a determining step of determining a humidity state of the internal space of the endoscope based upon the detected leakage current.

In order to achieve the above object, a humidity detecting device for detecting humidity in an internal space of an endoscope according to the present invention, comprising: a detecting device for detecting a leakage current of an imaging device and/or a peripheral circuit of the imaging device arranged in the internal space of the endoscope; and a determining device for determining a humidity state in the internal space of the endoscope based upon the leakage current detected by the detecting device.

Here, the endoscope includes an insertion part, a hand operation part, an LG (light guide) connector connected to the hand operation part via a universal cable, and it indicates a portion which is cleaned by a cleaning apparatus or is sterilized in an autoclave sterilizing apparatus. Further, the internal space of the endoscope indicates a space for housing built-in parts such as an imaging device inside the endoscope.

In the humidity detecting method and device for an endoscope according to the present invention, since leakage current of the imaging device and/or the peripheral circuit of the imaging device arranged in the internal space of the endoscope is detected and the humidity state of the internal space of the endoscope is determined based upon the leakage current detected, it is unnecessary to provide a special humidity sensor in the internal space of the endoscope. Further, since the humidity environment around the imaging device can be precisely estimated by detecting the leakage current of the imaging device and/or the peripheral circuit of the imaging device, damage of built-in parts housed in the internal space of the endoscope, particularly the imaging device which hates dampness can be prevented.

Further, since the imaging device is generally provided at the distal end portion of the internal space of the endoscope, the humidity state of the distal end portion of the internal space of the endoscope where it is difficult to arrange a humidity sensor additionally, can be estimated.

Thus, since the humidity state of the distal end portion of the endoscope can be reliably estimated without enlarging the diameter of the insertion part of the endoscope, the imaging device such as CCD or another electric part can be prevented from malfunctioning or being damaged due to dampness.

In order to achieve the above object, an endoscope apparatus according to the present invention, the endoscope apparatus including an endoscope and a dehumidifying device which dehumidifies an internal space of the endoscope, wherein the dehumidifying device comprises: the humidity detecting device according to claim 2; a dehumidifying unit for dehumidifying an internal space of the endoscope, the dehumidifying unit which is attachable and detachable to the endoscope; and a control unit for controlling the dehumidifying unit based upon a humidity detection result in the internal space of the endoscope detected by the humidity detecting device.

In the endoscope apparatus according to the present invention, since a dehumidification control mechanism including the humidity detecting device which detects the humidity of the interior of the endoscope based upon the leakage current of the imaging device and/or the peripheral circuit of the imaging device, the dehumidifying unit which dehumidifies the internal space of the endoscope, and the control unit which controls these parts is attachably and detachably provided to the endoscope, detection of humidity or dehumidification of the internal space of the endoscope can be performed by only mounting the dehumidification control mechanism without changing the structure or the shape of the endoscope itself largely.

In the endoscope apparatus according to the present invention, it is preferred that the dehumidifying unit a ventilation-type dehumidifying unit comprising: a gas-feeding unit for feeding air to the internal space of the endoscope; and a suction unit for suctioning the air fed.

This shows an example of a specific configuration of the dehumidifying device suitable to dehumidify the internal space of the endoscope. In this case, it is preferred to provide a humidity sensor which measures humidity of air suctioned by the suction unit. Thereby, double checking of humidity can be realized by humidity detection of the internal space of the endoscope based upon the leakage current and by humidity detection of the interior of the endoscope by the humidity sensor.

In the endoscope apparatus according to the present invention, it is preferred that the dehumidifying unit is a heating-type dehumidifying device comprising: a metal-made buckling-preventing wire which covers an outer periphery of a soft pipe conduit provided in the internal space of the endoscope; and a current-supplying device for applying a current to the buckling-preventing wire to cause the buckling-preventing wire to generate heat.

In many cases, the metal-made buckling-preventing wire covering an outer periphery of a soft pipe conduit is originally provided in the endoscope in order to prevent buckling of the soft pipe conduit due to use of the endoscope. Therefore, by heating the buckling-preventing wire to dehumidify the internal space of the endoscope, a dehumidification system for the internal space of the endoscope can be established utilizing the members originally provided in the endoscope maximally together with the humidity detection of the interior of the endoscope based upon the leakage current.

Thus, since new members for establishing the dehumidification system can be reduced, the endoscope apparatus can be made compact and the number of parts can be reduced.

According to the humidity detecting method and device for an endoscope to which the present invention is applied, the humidity state of the internal space of the endoscope can be estimated utilizing the leakage current of the imaging device and/or the peripheral circuit of the imaging device provided in the endoscope, and thus, it is unnecessary to provide a humidity sensor in the internal space of the endoscope additionally. Accordingly, the humidity state of the distal end portion of the endoscope can be reliably estimated without increasing the size of the hard part of the distal end portion of the endoscope.

Further, according to the endoscope apparatus to which the present invention is applied, the humidity state of the internal space of the endoscope can be estimated by making full use of the parts originally provided in the endoscope, and the humidity of the internal space of the endoscope can be detected or the internal space can be dehumidified by only mounting a humidity control device on the endoscope attachably and detachably.

According to the present invention, therefore, even if the humidity state of the internal space of the endoscope becomes high due to treatments in the cleaning apparatus or the autoclave sterilizing apparatus, an imaging device such as CCD or another electric part can be prevented from malfunctioning or being damaged.

### BRIEF DESCRIPTION OF THE DRAWINGS

FIG. 1 is a perspective view of an endoscope to which the present invention is applied;

FIG. 2 is a perspective view showing an end face of a distal end hard part at an insertion part of the endoscope shown in FIG. 1;

FIG. 3 is a sectional view for explaining built-in parts such as an imaging device housed in an interior of the distal end hard part;

FIG. 4 is a conceptual diagram of an endoscope apparatus composed of an endoscope and a dehumidifying device;

FIG. 5 is a perspective view showing a connection relationship between a dehumidifying device, and an LG connector and an electric connector;

FIG. 6 is a graph showing a relationship between humidity in an internal space of an endoscope and leakage current;

FIG. 7 is an explanatory block diagram for explaining steps of a humidity detecting method according to an embodiment of the present invention;

FIG. 8 is a graph for explaining leakage current in the humidity detecting method;

FIG. 9 is an explanatory block diagram for explaining dehumidifying steps performed by a dehumidifying device; and

FIG. 10 is a conceptual diagram for explaining another aspect of an endoscope apparatus.

### DETAILED DESCRIPTION OF THE PREFERRED EMBODIMENTS

Preferred embodiments of humidity detecting method and device of an endoscope, and an endoscope apparatus according to the present invention will be described in detail below with reference to the accompanying drawings.

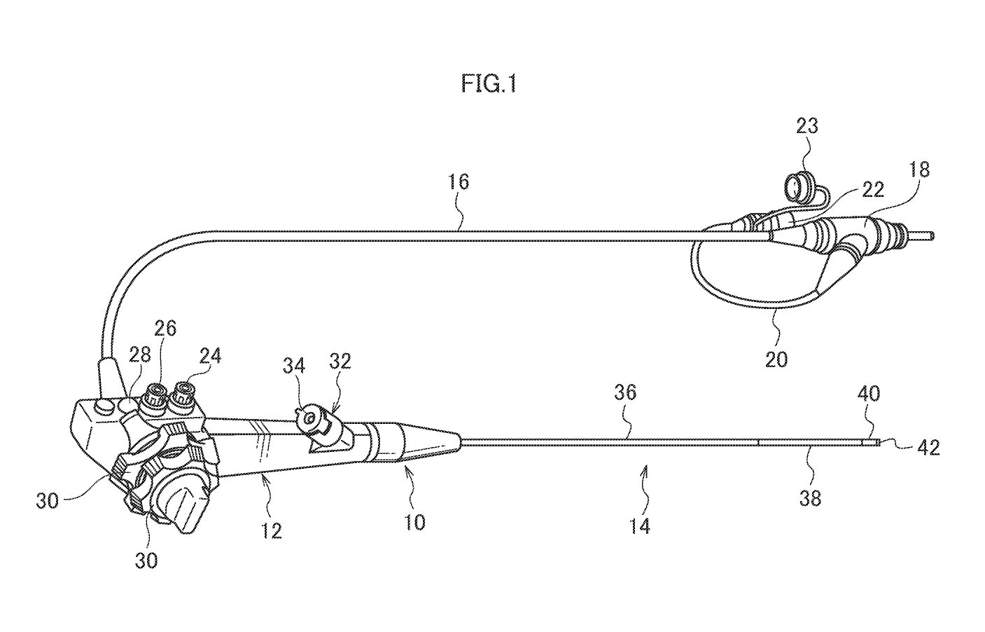

FIG. 1 is a perspective view of an endoscope 10 to which the present invention has been applied.

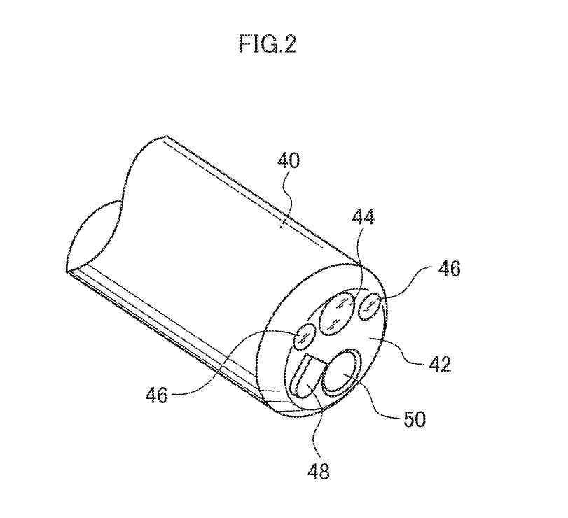

The endoscope 10 shown in FIG. 1 is provided with a hand operation part 12 which an operator grasps and an insertion part 14 provided in a coupled manner to the hand operation part 12 and inserted into a body cavity of a human. The hand operation part 12 is connected with a universal cable 16, and an LG connector 18 is provided at a distal end of the universal cable 16. The LG connector 18 is connected to a light source device (not shown) so that illumination light from the light source device is sent to illumination windows 46 and 46 shown in FIG. 2.

Further, the LG connector 18 shown in FIG. 1 is connected with an electric connector 22 via an electric cable 20, and the electric connector 22 is attachably and detachably connected to a processor (not shown). Reference numeral 23 shown in FIG. 1 denotes a cap for the electric connector 22, and the cap 23 is attached to the electric connector 22 when the electric connector 22 is washed in a cleaning apparatus.

The hand operation part 12 is provided with a gas-feeding/water-feeding button 24, a suction button 26 and a shutter button 28 arranged in parallel, and it is also provided with a pair of curvature-manipulation (bending-manipulation) knobs 30 and 30. Further, the hand operation part 12 is provided with a forceps insertion part 32, and a forceps plug 34 is attached to an opening end of the forceps insertion part 32.

The insertion part 14 is composed of a flexible pipe part 36, a bending part 38, and a distal end hard part 40 in this order from the side of the hand operation part 12. The curvature of bending part 38 is manipulated remotely according to rotation (revolution) of the curvature-manipulation knobs 30 and 30 of the hand operation part 12. Thereby, the distal end hard part 40 can be directed to a desired direction.

As shown in FIG. 2, an observation window 44, illumination windows 46 and 46, an gas-feeding/water-feeding nozzle 48, and a forceps port 50 are provided on a distal end face 42 of the distal end hard part 40.

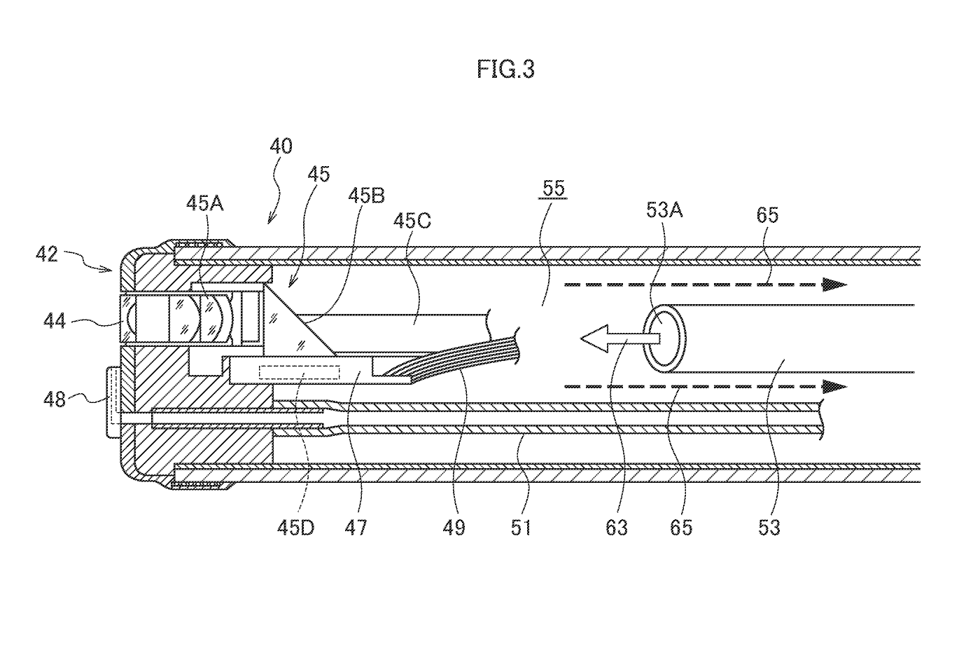

Further, as shown in FIG. 3, an imaging optical system 45 including a lens group 45A, a prism 45B, an imaging device 45D such as CCD (Charge-Coupled Device) or MOS (Metal-Oxide Semiconductor), and the like is disposed behind an objective lens constituting the observation window 44. The imaging device 45D is connected with an electric board 47, and an amplifier, a buffer, a peripheral circuit (not shown) such as a driving power source for the imaging device 45D, a signal processing circuit (not shown) for processing a signal of the imaging device 45D, and the like are mounted on the electric board 47. Power required for driving them is divided and supplied to the electric board 47 from a power-supplying part of a processor by connecting the electric connector 22 to the processor (not shown). Further, a signal cable 49 connected to the signal processing circuit passes through the insertion part 14, the hand operation part 12, the universal cable 16 and the like which are shown in FIG. 1 to extend up to the electric connector 22, and is connected to the processor.

An observation image taken in from the observation window 44 is focused on an light-receiving face of the imaging device 45D to be converted to an electric signal, and the electric signal is outputted to the processor via the signal cable 49 to be converted into a video signal. Thereby, the observation image is displayed on a monitor (not shown) connected to the processor.

A light-emitting end of a light guide 45C shown in FIG. 3 is arranged behind the illumination windows 46 and 46 shown in FIG. 2. The light guide 45C passes through the insertion part 14, the hand operation part 12, and the universal cable 16 shown in FIG. 1 so that an incident end of the light guide 45C is disposed in the LG (Light Guide) connector 18. Therefore, by connecting the LG connector 18 to the light source device, illumination light emitted from the light source device is transmitted to the illumination windows 46 and 46 via the light guide and emitted forward form the illumination windows 46 and 46.

The gas-feeding/water-feeding nozzle 48 shown in FIG. 3 communicates with a gas-feeding/water-feeding tube 51 made of resin and operated by the gas-feeding/water-feeding button 24, and the gas-feeding/water-feeding tube 51 communicates with a gas-feeding/water-feeding connector (not shown) of the LG connector 18 shown in FIG. 1. The gas-feeding/water-feeding connector is connected with a gas-feeding/water-feeding device (not shown), and air and water are supplied from the gas-feeding/water-feeding device. Therefore, by operating the gas-feeding/water-feeding button 24, air or water can be jetted toward the observation window 44 from the gas-feeding/water-feeding nozzle 48 shown in FIG. 2.

Further, as shown in FIG. 3, a blowing port 53A of a gas feeding tube for dehumidification 53 which is made of resin and blows air for dehumidifying an endoscope internal space 55 is disposed behind the imaging optical system 45. The gas feeding tube for dehumidification 53 passes through the insertion part 14, the hand operation part 12, the universal cable 16 and the like shown in FIG. 1 to extend up to the LG connector 18.

The forceps port 50 communicates with the forceps insertion part 32 shown in FIG. 1. Thus, by inserting a treatment tool such as a forceps from the forceps insertion part 32, the treatment tool can be guided out from the forceps port 50 shown in FIG. 2. Further, the forceps port 50 communicates with a suction valve (not shown) operated by the suction button 26 shown in FIG. 1, and the suction valve is further connected to a suction connector (not shown) of the LG connector 18. Therefore, by connecting a suction pump (not shown) to the suction connector and operating the suction valve with the suction button 26, waste material, residue or the like can be suctioned from the forceps port 50.

After the endoscope 10 thus configured has been used for medical diagnosis, an outer surface and inner pipe conduits (for example, a treatment tool insertion pipe conduit and the like) of the endoscope 10 are subjected to cleaning treatment by liquid such as a cleaning solution or rinsing solution in a cleaning apparatus, and the endoscope 10 is then put in a sterilizing package and subjected to sterilizing treatment by an autoclave sterilizing apparatus. In the cleaning treatment and the sterilizing treatment, if water, water vapor or the like enters the endoscope internal space 55, the imaging device 45D and the electric system which are built-in parts housed in the endoscope internal space 55 become easy to be damaged. Therefore, it is necessary to estimate the humidity state of the endoscope internal space 55 before the endoscope 10 is used and dehumidify the endoscope internal space 55 if the endoscope internal space 55 is in a high humidity state.

However, as shown in FIG. 3, the parts of the imaging optical system such as the imaging device 45D are densely arranged at the distal end portion of the endoscope internal space 55, and it is difficult to arrange a humidity sensor additionally without making the diameter of the insertion part 14 of the endoscope 10 large.

In the embodiment of the present invention, therefore, a dehumidifying device 57 which can detect humidity in the endoscope internal space 55 and can dehumidify the endoscope internal space 55 is attachably and detachably provided in the endoscope 10 without disposing a humidity sensor at the distal end portion of the endoscope internal space 55.

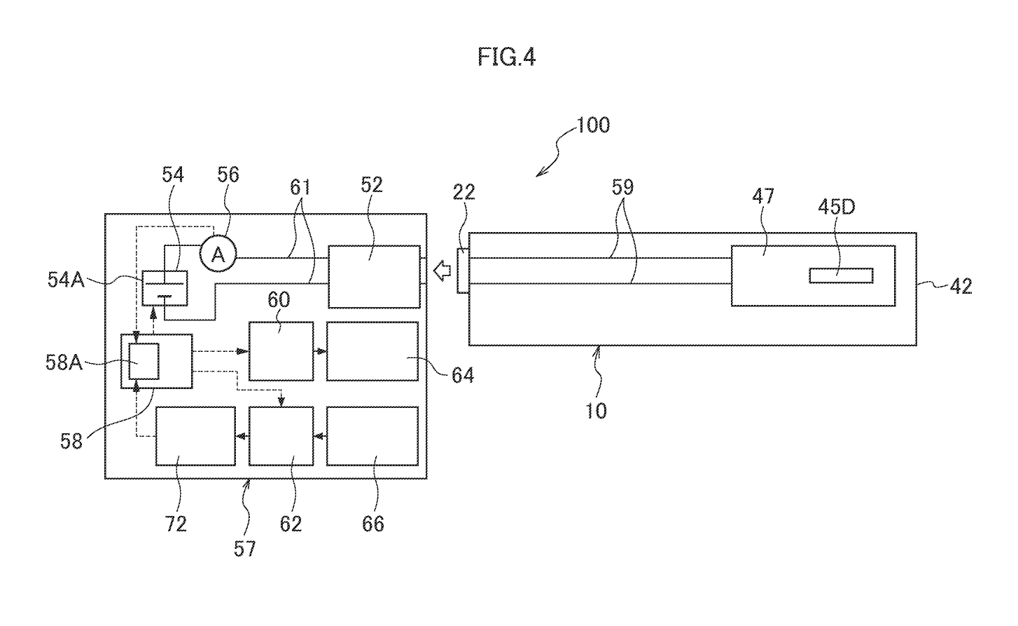

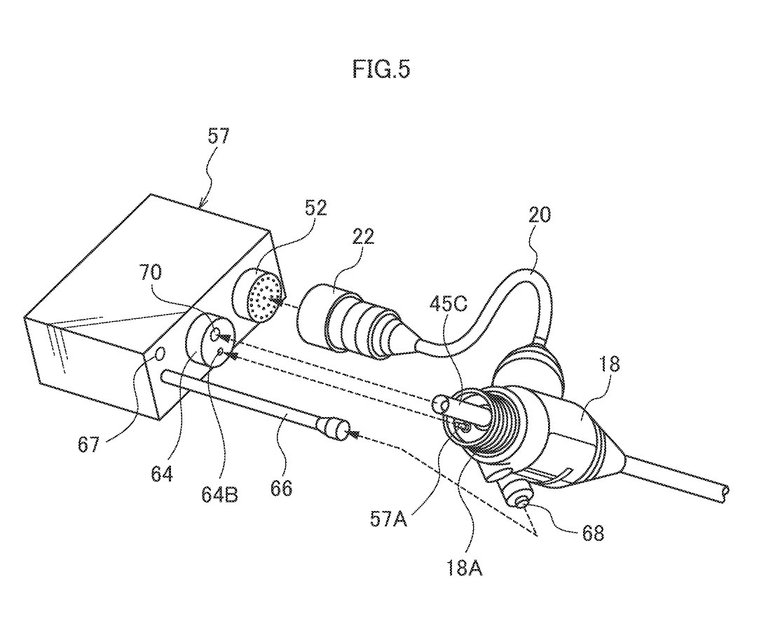

FIG. 4 and FIG. 5 are a block diagram and a perspective view for explaining a concept of an endoscope apparatus 100 including the endoscope 10 and the dehumidifying device 57.

As shown in FIG. 4, an electric cable 59 is provided to extend from the electric board 47 of the endoscope 10 up to the electric connector 22. On one hand, a connector 52 attachably and detachably connected with the electric connector 22 is provided in the dehumidifying device 57, and a power source circuit 61 is formed between the connector 52 and a power source part 54. Thereby, as shown in FIG. 4 and FIG. 5, when the electric connector 22 and the connector 52 are connected to each other, the power source part 54 of the dehumidifying device 57, and the imaging device 45D and the peripheral circuit of the electric board 47 are electrically connected to each other. Further, a voltage adjuster 54A is housed in the power source part 54, so that a voltage can be adjusted according to an instruction from a microcomputer 58 (control device).

The power source circuit 61 of the dehumidifying device 57 is provided with a current measuring part 56, and a current value measured by the current measuring part 56 is outputted to a determining part 58A of the microcomputer 58. The determining part 58A receives a relational expression showing a relationship between the humidity of the endoscope internal space 55, and the leakage currents of the imaging device 45D and the peripheral circuit of the electric board 47.

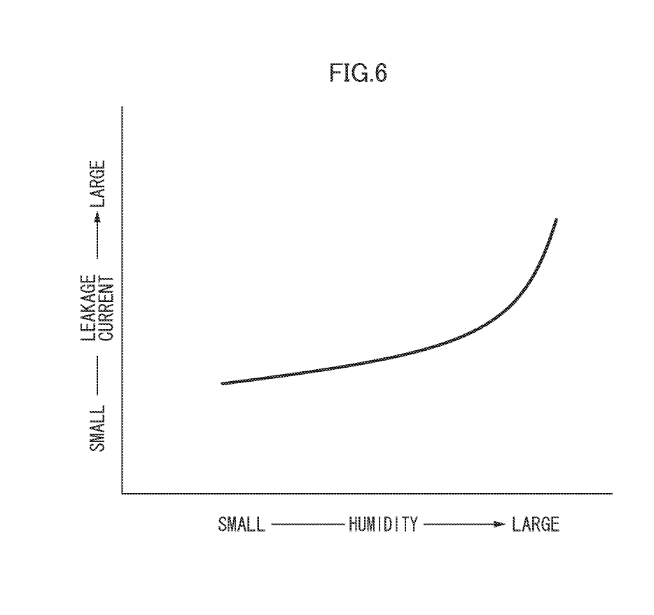

FIG. 6 is a graph showing a relationship between the humidity of the endoscope internal space 55, and the leakage currents of the imaging device 45D and the peripheral circuit of the electric board 47, from which a positive correlative relationship where the leakage currents from the imaging device 45D and the peripheral circuit of the electric board 47 increases according to increase of the humidity of the endoscope internal space 55 is found. Thus, by detecting the leakage currents of the imaging device 45D and the peripheral circuit of the electric board 47 by the current measuring part 56 and determining the magnitude (amount) of the leakage current detected by the current measuring part 56 by the determining part 58A, the humidity state in the endoscope internal space 55, particularly at the distal end portion of the endoscope can be estimated.

Further, as shown in FIG. 4, a gas-feeding pump 60 and a suction pump 62 are provided in the dehumidifying device 57 and driving of them is controlled by the microcomputer 58. The gas-feeding pump 60 has a gas-feeding connector 64 and the suction pump 62 has a suction connector 66. As shown in FIG. 5, by attachably and detachably connecting the gas-feeding connector 64 and a connection port 18A of the LG connector 18, communication with a proximal end port 57A (see FIG. 5) of the gas-feeding tube for dehumidification 53 extending from the distal end hard part 40 up to the LG connector 18 is achieved via a gas-feeding port 64B (see FIG. 5) of the gas-feeding connector 64.

Incidentally, an insertion port 70 formed in the gas-feeding connector 64 is an insertion port for the light guide 45C, and it houses the light guide 45C such that the light guide 45C does not block an operation performed when the gas-feeding connector 64 and the connection port 18A of the LG connector 18 are connected to each other.

Further, the suction pump 62 is attachably and detachably coupled to a suction port 68 (see FIG. 5) formed in the LG connector 18 via the suction connector 66 (see FIG. 5). The suction port 68 communicates with the endoscope internal space 55.

Thereby, air fed from the gas-feeding pump 60 is blown out toward the distal end of the endoscope internal space 55 via the gas-feeding connector 64 and the gas-feeding tube for dehumidification 53 as shown by an arrow 63 in FIG. 3. The blown-out air flows in the endoscope internal space 55 to the side of the LG connector through the outside of the gas-feeding tube for dehumidification 53 as shown by broken arrows 65 in FIG. 3 and is suctioned into the suction pump 62 via the suction connector 66.

Further, the suction pump 62 is provided with a humidity sensor 72 which measures the humidity of the air suctioned from the endoscope internal space 55, and the humidity data of suctioned air measured by the humidity sensor 72 is outputted to the determining part 58A of the microcomputer 58.

Next, a humidity detecting method for detecting humidity of the endoscope internal space 55 using the dehumidifying device 57 of the endoscope apparatus 100 will be described.

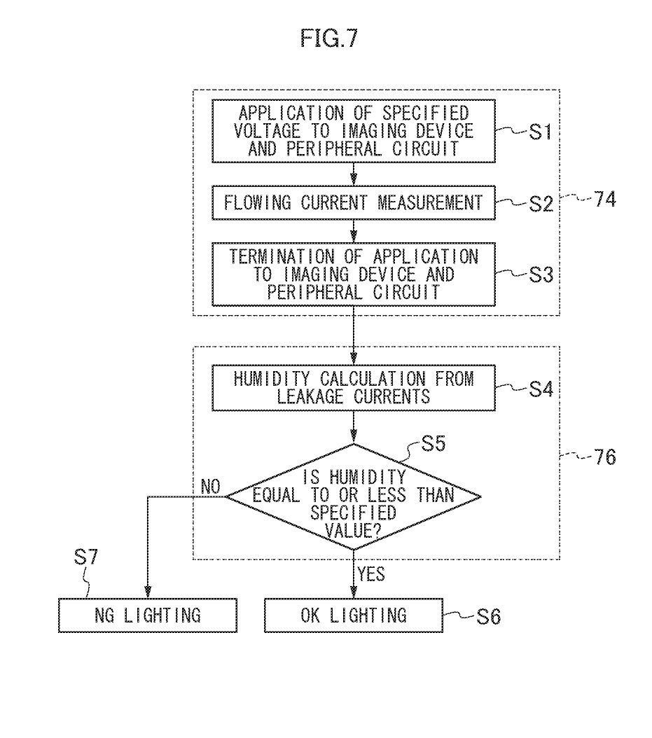

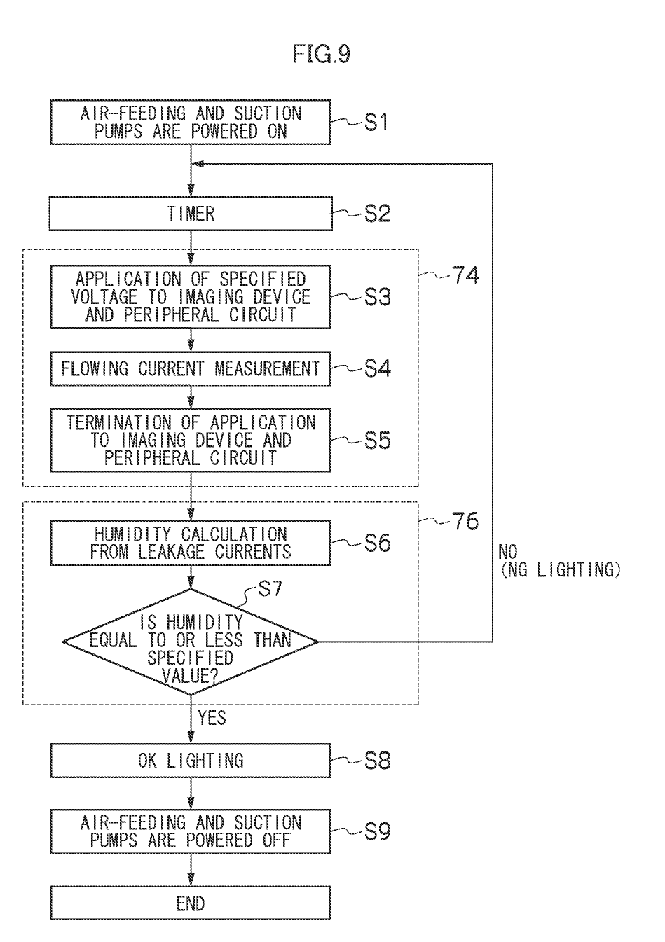

Steps shown in FIG. 7 are performed according to a program stored in the microcomputer 58. Incidentally, the steps shown in FIG. 7 correspond to the case where only a device regarding humidity detection of the dehumidifying device 57 has been driven, and steps which further include a device regarding dehumidification will be described in FIG. 9.

First, as shown in FIG. 5, the respective connectors 52, 64 and 66 of the dehumidifying device 57 are connected to the electric connector 22 and the LG connector 18 of the endoscope 10 and a leakage current detecting process 74 of Step 1 to Step 3 is performed.

That is, as shown in FIG. 7, current is fed from the power source part 54 of the dehumidifying device 57 to the imaging device 45D and the peripheral circuit of the electric board 47 of the endoscope 10, thereby applying a specified voltage to the power source driving circuit (Step 1). A current-supply time may be a time period where leakage current can be detected, but it is preferred that the current-supply time is set as short as possible.

The leakage current is detected by measuring a current value obtained when the specified voltage is applied to the imaging device 45D and the peripheral circuit of the electric board 47 by the current measuring part 56 of the dehumidifying device 57 (Step 2), and current-supply to the imaging device 45D and the peripheral circuit of the electric board 47 is stopped after the detection (Step 3). The time period of application of the specified voltage to the imaging device 45D and the peripheral circuit of the electric board 47 is set by a timer function of the microcomputer 58.

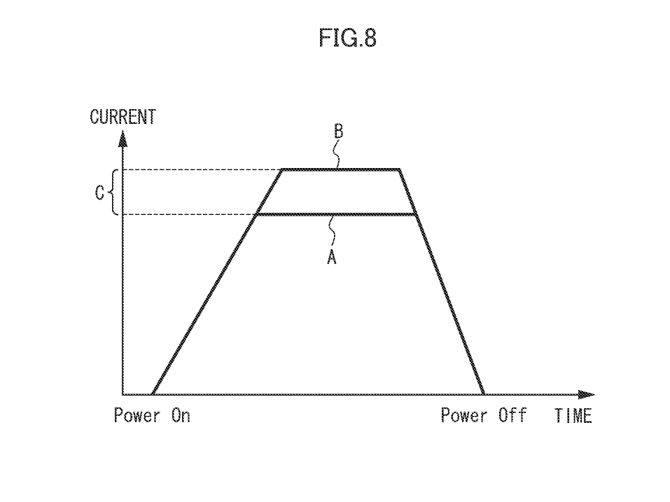

FIG. 8 is a graph showing whether a current value obtained when the power source part 54 is turned on and voltage applied to the imaging device 45D and the peripheral circuit of the electric board 47 is gradually increased to reach the specified voltage is a normal voltage value A (i.e., a current value which does not include a current value due to the leakage current) or an abnormal current value B higher than the normal current value due to the leakage current. Thus, a difference in current value obtained by subtracting the normal current value A from the abnormal current value B becomes a value C of the leakage current.

Therefore, when the current value measured by the current measuring part 56 is the normal current value A, it is determined that the humidity in the endoscope internal space 55 is low, and that even if the endoscope 10 is used, the imaging device 45D or the electric system is not damaged.

However, when the current value is the abnormal current value B, it is determined that the humidity in the endoscope internal space 55 is high, and that there is a possibility that when the endoscope 10 is used, the imaging device 45D or the electric system is damaged. Therefore, even in the leakage current detecting process 74, there is a possibility that when the voltage is raised up to the specified voltage at once in a state where the humidity in the endoscope internal space 55 is high, large current flowing in the imaging device 45D may damage the imaging device 45D. For this reason, it is preferred that the microcomputer 58 controls the voltage adjustor 54A to gradually raise the voltage up to the specified voltage as shown in FIG. 8.

As shown in FIG. 7, after the leakage current detecting process 74 is completed, control proceeds to a determining process 76 for determining the humidity state in the endoscope internal space 55 by the determining part 58A of the microcomputer 58.

That is, the determining part 58A of the microcomputer 58 calculates humidity based upon the leakage current value from the relationship between the leakage current and the humidity shown in FIG. 6 (Step 4) and determines whether the humidity is equal to or less than the specified value (Step 5).

Here, the specified value means the humidity under which the imaging device 45D and the peripheral circuit of the electric board 47 operate normally even if current is fed from the power source part of the processor for using the endoscope. When the humidity is equal to or less than the specified value (YES), a lamp 67 provided on the dehumidifying device 57 is lightened so as to indicate OK (Step 6). When the humidity exceeds the specified value (NO), the lamp is lightened so as to indicate NG (Step 7). For example, such a configuration can be adopted that two red and blue lamps are provided so that the blue lamp is lightened in the case of YES, while the red lamp is lightened in the case of NO. Alternatively, such a configuration can be adopted that one lamp is used, and the lamp is lightened in the case of YES, while the lamp is blinked in the case of NO.

Thus, according to the endoscope apparatus 100 of the embodiment of the present invention, since the humidity in the endoscope internal space 55 is detected utilizing the leakage currents of the imaging device 45D and the peripheral circuit of the electric board 47 which are originally provided in the endoscope 10, it is unnecessary to provide a humidity sensor in the endoscope internal space 55 additionally. Thereby, the humidity state in the endoscope internal space 55, particularly at the distal end portion thereof can be estimated reliably without enlarging the diameter of the insertion part 14 of the endoscope 10.

FIG. 9 is a flowchart for explaining steps for performing dehumidification in the endoscope internal space 55 using the dehumidifying device 57. For example, the steps are performed after the cleaning treatment in the cleaning apparatus and a sterilizing treatment in the autoclave sterilizing apparatus have been performed. The steps shown in FIG. 9 are performed according to a program stored in the microcomputer 58.

As shown in FIG. 9, the gas-feeding pump 60 and the suction pump 62 in the dehumidifying apparatus 57 are turned ON (Step 1).

Next, a timer (not shown) of the microcomputer 58 starts to operate (Step 2) and a leakage current detecting process 74 from Step 3 to Step 5 is performed. The leakage current detecting process 74 is similar to Steps 1 to 3 shown in FIG. 7, and explanation thereof is omitted. The timer sets a time period of application of the specified voltage to the imaging device 45D and the peripheral circuit of the electric board 47 in the leakage current detecting process 74.

Next, a determining process 76 including Step 6 and Step 7 is performed, the humidity is calculated from the magnitude (amount) of the leakage current detected in the leakage current detecting process 74, and whether the humidity is equal to or less than the specified value is determined. The determining process 76 is similar to Steps 4 and 5 shown in FIG. 7 and explanation thereof is omitted.

Next, in the determining process 76, when the humidity exceeds the specified value (NO), the lamp 67 is lightened so as to indicate NG and control returns back to Step 2, where the leakage current detecting process 74 is performed again. Operation to return back to Step 2 is repeated until the humidity in the determining process 76 becomes the specified value or less.

When the humidity in the determining process 76 becomes the specified value or less, the lamp 67 is lightened so as to indicate OK (Step 8), and the gas-feeding pump 60 and the suction pump 62 are tuned OFF (Step 9).

Thought not illustrated at steps in FIG. 9, it is further preferred that the humidity of the suctioned air suctioned from the endoscope internal space 55 by the suction pump 62 is periodically measured by the humidity sensor 72. That is, the humidity measurement utilizing the leakage currents of the imaging device 45D and the peripheral circuit of the electric board 47 makes it possible to estimate the humidity state in the distal end portion of the endoscope internal space 55 housing the imaging optical system such as the imaging device 45D and the humidity measurement of the suctioned air performed by the humidity sensor 72 makes it possible to estimate an average humidity state in the endoscope internal space 55. Thereby, double checking of the humidity detection in the endoscope internal space 55 based upon the leakage current and the humidity detection in the endoscope internal space performed by the humidity sensor 72 can be performed.

Therefore, it is further preferred that when both of the humidity measurement results based upon the leakage current and the humidity measurement by the humidity sensor 72 are equal to the specified value or less, the lamp is lightened to indicate OK fashion. However, it is most important to protect the imaging device 45D and the peripheral circuit of the electric board 47 or the like from dampness or moisture, and it is possible to use the humidity measurement by the humidity sensor 72 secondarily.

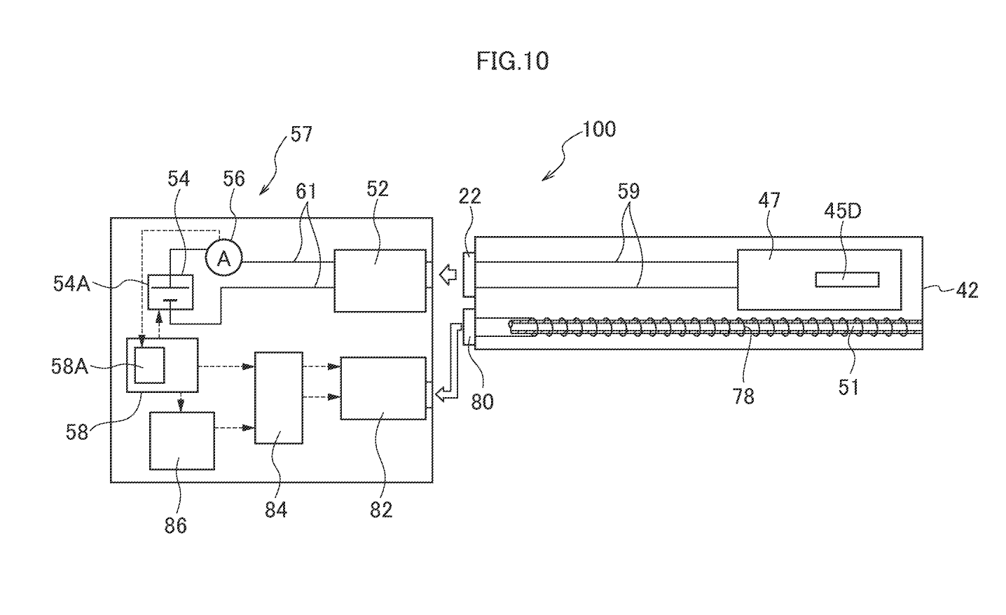

FIG. 10 shows another aspect of the endoscope apparatus 100. The endoscope apparatus 100 is configured to dehumidify the endoscope internal space 55 by causing a metal-made buckling-preventing wire to generate heat, the metal-made buckling-prevention wire which covers an outer periphery of a soft pipe conduit provided in the endoscope internal space 55.

As the soft pipe conduit, for example, at least a pipe conduit which is provided in the endoscope internal space 55 on the side of the insertion part 14 can be preferably used among a treatment tool introduction pipe conduit, a gas-feeding/water-feeding pipe conduit and a suction pipe conduit which are provided in the endoscope internal space 55 on the side of the insertion part 14, and a gas-feeding/water-feeding pipe conduit and a suction pipe conduit which are provided in the endoscope internal space 55 on the side of the universal cable.

Here, the case that the gas-feeding/water-feeding tube 51 (see FIG. 3) is used as the soft pipe conduit will be described. Incidentally, explanation of same members as described in the endoscope apparatus 100 in FIG. 4 is omitted in order to avoid duplicative explanation.

As shown in FIG. 10, a metal-made buckling-preventing wire 78 is wound on an outer periphery of the gas-feeding/water-feeding tube 51 arranged in the endoscope internal space 55 of the endoscope 10. Both end portions of the buckling-preventing wire 78 are connected to a heating connector 80 provided in the LG connector 18.

On one hand, a connector for a heating power source 82 which is attachably and detachably connected with the heating connector 80 of the endoscope 10 is provided in the dehumidifying device 57, and the connector for a heating power source 82 is electrically wired to a heating power source 86 via a heating temperature adjuster 84. Further, driving of the heating temperature adjuster 84 and the heating power source 86 are controlled by the microcomputer 58.

Thereby, when the respective connectors of the dehumidifying device 57 and the endoscope 10 are connected to each other and the heating power source 86 is turned (powered) ON, the metal-made buckling-preventing wire 78 wound on the outer periphery of the gas-feeding/water-feeding tube 51 can generate heat to perform dehumidification of the endoscope internal space. The degree of heat generation is adjusted by the heating temperature adjuster 84.

The dehumidification of the endoscope internal space 55 performed by the dehumidifying device 57 shown in FIG. 10 can be similarly performed by replacing the “gas-feeding and suction pumps are turned ON” at Step 1 shown in FIG. 9 with “heating power source is turned ON”.

According to the endoscope apparatus shown in FIG. 10, the humidity in the endoscope internal space 55 can be measured utilizing the leakage current of the imaging device 45D or the peripheral circuit of the electric board 47 and the dehumidification in the endoscope internal space 55 can be performed utilizing the buckling-preventing wire 78.

Therefore, since the members originally provided in the endoscope 10 can be utilized maximally, reconstruction of the endoscope 10 is hardly required. Thus, since new members required for constructing the dehumidifying system can be reduced, the endoscope apparatus 100 can be made compact, and the number of parts can be reduced.

Incidentally, the embodiment is configured so as to measure the leakage currents of the imaging device 45D and the peripheral circuit of the electric board 47, but such a configuration can be adopted that one of the leakage currents of the imaging device 45D and the peripheral circuit of the electric board 47 is measured.

What is claimed is:

1. A humidity detecting method for detecting humidity in an internal space of an endoscope, comprising:
a leakage current detecting process for detecting a leakage current of an imaging device and/or a peripheral circuit of the imaging device arranged in the internal space of the endoscope; and
a determining process for determining a humidity state in the internal space of the endoscope based upon the leakage current detected.

2. A humidity detecting device for an endoscope, the humidity detecting device for detecting humidity in an internal space of an endoscope, comprising:
a detecting device for detecting a leakage current of an imaging device and/or a peripheral circuit of the imaging device arranged in the internal space of the endoscope; and
a determining device for determining a humidity state in the internal space of the endoscope based upon the leakage current detected by the detecting device.

3. An endoscope apparatus including an endoscope and a dehumidifying device which dehumidifies an internal space of the endoscope, wherein
the dehumidifying device comprises:
the humidity detecting device according to claim 2;
a dehumidifying unit for dehumidifying an internal space of the endoscope, the dehumidifying unit which is attachable and detachable to the endoscope; and
a control unit for controlling the dehumidifying unit based upon a humidity detection result in the internal space of the endoscope detected by the humidity detecting device.

4. The endoscope apparatus according to claim 3, wherein
the dehumidifying unit is a ventilation-type dehumidifying unit comprising:
a gas-feeding unit for feeding air to the internal space of the endoscope; and
a suction unit for suctioning the air fed.

5. The endoscope apparatus according to claim 4, further comprising a humidity sensor which measures a humidity of the air suctioned by the suction unit.

6. The endoscope apparatus according to claim 3, wherein
the dehumidifying unit is a heating-type dehumidifying device comprising:
a metal-made buckling-preventing wire which covers an outer periphery of a soft pipe conduit provided in the internal space of the endoscope; and
a current-supplying device for applying a current to the buckling-preventing wire to cause the buckling-preventing wire to generate heat.

7. The endoscope apparatus according to claim 6, wherein
the soft pipe conduit is at least a pipe conduit which is provided in the internal space of the endoscope on a side of an insertion part, among a treatment tool introduction pipe conduit, a gas-feeding/water-feeding pipe conduit and a suction pipe conduit which are provided in the internal space on the side of the insertion part, and a gas-feeding/water-feeding pipe conduit and a suction conduit which are provided in the internal space of the endoscope on a side of universal cable.
